(19) United States
(12) Patent Application Publication
(10) Pub. No.: US 2025/0339149 A1
(43) Pub. Date: Nov. 6, 2025

(54) AUTOMATIC LIGATURE-CUTTING LIGATION DEVICE AND LIGATION SYSTEM

(71) Applicant: MEDLEAD MEDICAL TECHNOLOGY CO., LTD., SHENZHEN (CN)

(72) Inventor: Guojun HE, SHENZHEN (CN)

(73) Assignee: MEDLEAD MEDICAL TECHNOLOGY CO., LTD., SHENZHEN (CN)

(21) Appl. No.: 19/268,539

(22) Filed: Jul. 14, 2025

Related U.S. Application Data
(63) Continuation-in-part of application No. PCT/CN2023/107660, filed on Jul. 17, 2023.

(30) Foreign Application Priority Data
Jun. 30, 2023 (CN) 202321699038.3

(51) Int. Cl. A61B 17/12; A61B 17/00; A61B 90/00

(52) U.S. Cl. CPC A61B 17/12; A61B 17/00234; A61B 90/39; A61B 2017/00243; A61B 2017/00292; A61B 2017/00353; A61B 2017/00358; A61B 2017/00367; A61B 2017/00477; A61B 2017/00862; A61B 2017/00867; A61B 2090/3966

(57) ABSTRACT

An automatic ligature-cutting ligation device and a ligation system are provided. The ligation device includes: a handle assembly, including a first handle and a second handle detachably connected to each other; a catheter assembly, an end of the catheter assembly being connected to the first handle; a snare, disposed at another end of the catheter assembly, where a slit is defined in an inner surface of the snare; a ligature, penetrating the first handle and the catheter assembly, where an end of the ligature is connected to the second handle, another end of the ligature has a loop capable of contracting in a forward direction and stopping in a reverse direction, and the loop is pre-mounted in the snare; and a ligature-cutting assembly, disposed in the second handle.

### CROSS REFERENCE TO RELATED APPLICATIONS

The present application a continuation-in-part application of International (PCT) Patent Application No. PCT/CN2023/107660, filed on Jul. 17, 2023, which claims priority of Chinese patent Application No. 202321699038.3, filed on Jun. 30, 2023, both of which are hereby incorporated by reference in their entirety.

### TECHNICAL FIELD

The present disclosure relates to the technical field of medical devices, and in particular to an automatic ligature-cutting ligation device and a ligation system.

### BACKGROUND

A left atrial appendage (LAA) is a long and narrow tubular blind chamber. The LAA may have an irregular shape with multiple twists and turns. A size of the LAA may be similar to that of a thumb, and an opening size of the LAA may be in a range from 10 mm to 40 mm. The LAA may include abundant pectinate muscles and muscle trabecular, such that blood flow in the LAA may be prone to vortex formation and reduced velocity, which may cause the LAA to be a common site for thrombus formation.

A patient with atrial fibrillation (AF) may have a significantly increased risk of stroke. Studies indicate that 60% of cardiogenic thrombi in patients with rheumatic heart disease complicated with the AF come from the LAA, and 91% of strokes caused by non-valvular AF are caused by thrombi in the LAA. Therefore, removing the LAA may help reduce left atrial thrombus and stroke events.

At present, clinical treatment methods include oral anticoagulant drugs, radiofrequency ablation, percutaneous left atrial appendage closure, and surgical LAA resection. However, regardless of the method, the clinical effect is not very satisfactory. At present, clinical treatment methods may include: oral anticoagulant drugs, radiofrequency ablation, percutaneous LAA closure, and surgical LAA resection. However, regardless of any one of the methods, a clinical effect is not very satisfactory.

Therefore, a LAA ligation is emerged. Accordingly, matching medical devices may also be developed.

### SUMMARY OF THE DISCLOSURE

According to a first aspect, some embodiments of the present disclosure provide an automatic ligature-cutting ligation device. The automatic ligature-cutting ligation device includes: a handle assembly, including a first handle and a second handle detachably connected to each other; a catheter assembly, an end of the catheter assembly being connected to the first handle; a snare, disposed at another end of the catheter assembly, where a slit is defined in an inner surface of the snare; a ligature, penetrating the first handle and the catheter assembly, where an end of the ligature is connected to the second handle, another end of the ligature has a loop capable of contracting in a forward direction and stopping in a reverse direction, and the loop is pre-mounted in the snare; and a ligature-cutting assembly, disposed in the second handle; where in a case where the second handle is separated from the first handle and pulled out in a direction away from the first handle, the ligature is driven to pull out in the direction away from the first handle, and the loop is driven to pull out of the snare from the slit to an inner side enclosed by the snare and contract in the forward direction; and in a case where the second handle is separated from the first handle and pulled out in the direction away from the first handle until a preset pulling force is reached, the ligature-cutting assembly is driven to move and cut the ligature.

According to a second aspect, some embodiments of the present disclosure provide an automatic ligature-cutting ligation device. The automatic ligature-cutting ligation device includes: a first handle; a second handle, detachably connected to the first handle; a catheter assembly, an end of the catheter assembly being connected to the first handle; a snare, disposed at another end of the catheter assembly, where a slit is defined in an inner surface of the snare; a ligature-cutting assembly, disposed in the second handle; and a ligature, penetrating the first handle and the catheter assembly, where an end of the ligature close to the snare is configured to form a unidirectional contraction loop, the loop is disposed in the snare, an end of the ligature away from the snare is connected to the ligature-cutting assembly; where in a case where the second handle is configured to move away from the first handle, the loop is driven to move out of the snare from the slit and contract unidirectionally; and in a case where the loop contracts unidirectionally until a snare area of the loop reaches a preset snare area, the ligature is configured to drive the ligature-cutting assembly to move towards the ligature until the ligature-cutting assembly reaches a ligature-cutting node and cuts the ligature.

According to a third aspect, some embodiments of the present disclosure provide a ligation system. The ligation system includes an automatic ligature-cutting ligation device, including: a handle assembly, including a first handle and a second handle detachably connected to each other; a catheter assembly, an end of the catheter assembly being connected to the first handle; a snare, disposed at another end of the catheter assembly, where a slit is defined in an inner surface of the snare; a ligature, penetrating the first handle and the catheter assembly, where an end of the ligature is connected to the second handle, another end of the ligature has a loop capable of contracting in a forward direction and stopping in a reverse direction, and the loop is pre-mounted in the snare; and a ligature-cutting assembly, disposed in the second handle; and an outer sheath, at least a part of the automatic ligature-cutting ligation device passing through the outer sheath, and the outer sheath being configured to deliver an end of the automatic ligature-cutting ligation device where the snare is disposed into a heart; where in a case where the second handle is separated from the first handle and pulled away from the first handle, the ligature is driven to be moved away from the first handle, and the loop is driven to be pulled out of the snare from the slit to an inner side enclosed by the snare and unidirectionally contract on the target tissue; and in a case where the second handle is separated from the first handle and pulled away from the first handle until a preset pulling force is reached, the ligature-cutting assembly is driven to move and cut the ligature.

### BRIEF DESCRIPTION OF THE DRAWINGS

In order to more clearly describe the technical solutions in the embodiments of the present disclosure or the related art, the drawings that need to be used in the description of the embodiments or the related art will be briefly described in the following. Apparently, the drawings in the following description are only some embodiments of the present disclosure. For those skilled in the art, other drawings can be obtained based on these drawings without creative work.

FIG. 1 is a schematic structural view of an automatic ligature-cutting ligation device according to some embodiments of the present disclosure.

FIG. 2 is a schematic structural view of a handle assembly in a separated state according to some embodiments of the present disclosure.

FIG. 3 is an inner schematic structural view of a ligature-cutting assembly mounted in a second handle shown in FIG. 1 according to some embodiments of the present disclosure.

FIG. 4 is an exploded structural schematic view of a part of a catheter assembly and a snare in FIG. 1

FIG. 5 is a schematic structural view of a ligature-cutting assembly mounted on the second handle in FIG. 1 according to another embodiment of the present disclosure.

FIG. 6 is a schematic structural view of a sliding member according to some embodiments of the present disclosure.

FIG. 7 is a schematic structural view of a snare, a part of a ligature, and a connecting tube according to some embodiments of the present disclosure.

FIG. 8 is a cross-sectional structural view of the snare shown in FIG. 1 along a radial direction of the snare.

FIG. 9 is a schematic structural view of a ligation system according to some embodiments of the present disclosure.

### REFERENCE SIGNS IN DRAWINGS

100, automatic ligature-cutting ligation device; 11, handle assembly; 12, catheter assembly; 13, ligature-cutting assembly; 200, outer sheath; 300, ligation system;
1, first handle;
2, second handle; 20, grip portion; 28, engagement portion; 201, first accommodating chamber; 202, second accommodating chamber; 203, first limiting shaft; 204, second limiting shaft; 208, through hole; 209, side wall; 21, sliding member; 210, base portion; 211, extension portion; 2111, first extension portion; 2112, second extension portion; 212, fixing pillar; 213, protrusion; 2131, groove;
29, connecting rod member; 22, first connecting rod; 221, connecting shaft; 23, second connecting rod;
231, first rod portion; 232, second rod portion; 233, rotational mounting portion; 234, connecting hole;
24, elastic member; 25, ligature-cutting blade; 26, opening; 27, ligature-cutting base; 270, notch;
3, snare; 31, radiopaque tungsten wire; 32, skeleton ring; 33, first channel; 34, second channel; 301, slit;
4, ligature; 41, loop; 42, one-way knot;
5, heat-shrinkable sleeve;
6, flexible catheter;
7, connection tube; 71, radiopaque coil spring.

### DETAILED DESCRIPTION

The technical solutions in the embodiments of the present disclosure will be clearly and completely described below with reference to the drawings in the embodiments of the present disclosure. It is apparent that the described embodiments are a part of the embodiments of the present disclosure rather, than all the embodiments. All other embodiments obtained by those of ordinary skill in the art without creative efforts based on the embodiments in the present disclosure shall fall within the protection scope of the present disclosure.

It should be understood that in a case where used in the description and the appended claims, terms “comprise/comprising” and “include/including” indicate the presence of the described features, integers, steps, operations, elements, and/or components, but do not exclude the presence or addition of one or more other features, integers, steps, operations, elements, components, and/or combinations thereof.

It should also be understood that terms used herein in the description of the embodiments of the present disclosure is for the purpose of describing particular embodiments only and is not intended to limit the embodiments of the present disclosure. As used in the description of the embodiments of the present disclosure and the appended claims, singular forms “a”, “an” and “the” are intended to include plural forms unless the context clearly indicates otherwise.

It should be further understood that a term “and/or” used in the description of the present disclosure and the appended claims may be referred to any combinations and all possible combinations of one or more of the listed item and include the combinations. For example, A and/or B, which can mean: A alone, both A and B, and B alone. In addition, the character “/” in this document generally indicates that the before and after associated objects are in an “or” relationship.

Terms “first”, “second” and “third” herein are used for descriptive purposes only and shall not be interpreted as indicating or implying relative importance or implicitly specifying the number of indicated technical features. Therefore, a feature defined by the “first”, “second”, or “third” may explicitly or implicitly include at least one such feature. All directional indications in the present disclosure (such as up, down, left, right, front, rear, . . . ) are used only to explain relative position relationship, movement, and the like, between components at a particular posture (as shown in the drawings). In a case where the posture is changed, the directional indications may change accordingly. In addition, terms “include” and “have” and any variations thereof are intended to cover non-exclusive inclusion. For example, a process, a method, a system, a product, or an apparatus including a series of operations or units is not limited to the listed operations or units, but may further include operations or units that are not listed, or include other operations or units that are inherent to the process, the method, the product, or the apparatus.

In the related art, a left atrial appendage (LAA) ligation technology is configured to close the LAA to reduce left atrial thrombus and stroke events. When using a LAA ligation device, a pre-formed ligature loop may be typically delivered to a target surgical area by utilizing a delivery tool, and an operator may pull the loop to tighten and complete the ligation by applying external hand force. During the above operation process, a judgment of whether the ligation is completed or firm mainly depends on a visual judgment and hand feeling of an operating doctor, which is prone to an operational error or deviation.

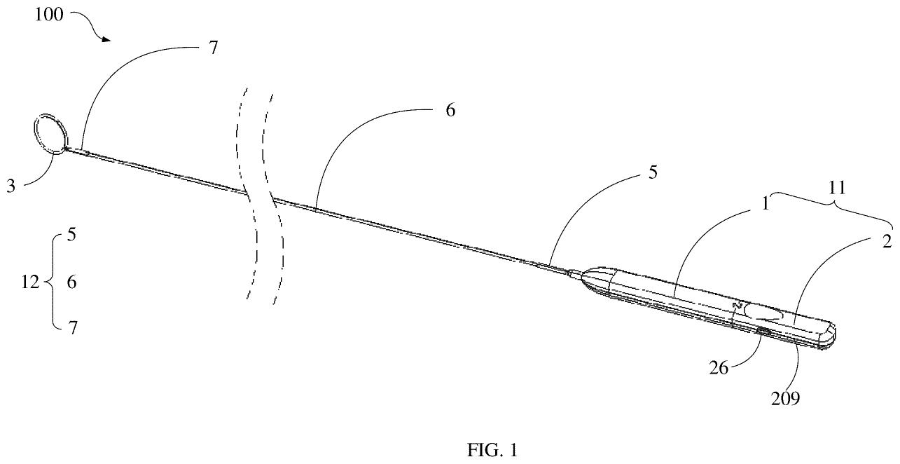

Therefore, an automatic ligature-cutting ligation device may be provided by some embodiments of the present disclosure, such that it may be possible to reduce the reliance on the visual judgment and tactile requirements, thereby improving operation accuracy and convenience, and reducing difficulty. As shown in FIGS. 1-4, an automatic ligature-cutting ligation device 100 may be provided by some embodiments of the present disclosure. The automatic ligature-cutting ligation device 100 may include a handle assembly 11, a catheter assembly 12, a snare 3, and a ligature 4.

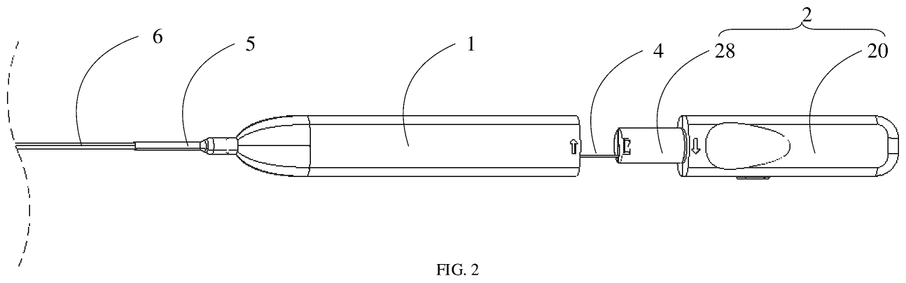

As shown in FIGS. 1-2, the handle assembly 11 may include a first handle 1 and a second handle 2 detachably connected to each other. An end of the catheter assembly 12 may be connected to the first handle 1, and another end of the catheter assembly 12 may be connected to the snare 3. The second handle 2 may be disposed at a side of the first handle 1 away from the snare 3. A receiving channel may be defined in the snare 3, and a slit may be defined in/at an inner surface of the snare 3. The ligature 4 may penetrate the first handle 1 and the catheter assembly 12. An end of the ligature 4 may be connected to the second handle 2. Another end of the ligature 4 may be a loop 41, and the loop 41 may be capable of contracting/tightening in a forward direction and stopping in a reverse direction. The loop 41 may be configured to be sleeved on and fastened to a target tissue during a ligation operation. The expression of “the loop 41 may be capable of contracting in the forward direction and stopping in the reverse direction” may be understood as: the loop 41 may be a unidirectional contraction loop, i.e., the loop 41 may only contract an inner diameter thereof in the forward direction to reduce a snare area of the loop 41, but the loop 41 cannot expand the inner diameter thereof in the reverse direction, so as to prevent the snare area of the loop 41 from increasing. When using the loop 41, the inner diameter of the loop 41 may only be gradually contracted toward the sleeved target tissue in the forward direction, and the inner diameter of the loop 41 may not be expanded in the reverse direction. Therefore, the loop 41 may be firmly sleeved on the target tissue without separating from the target tissue. The target tissue mentioned in the embodiments of the present disclosure may be referred to a tissue to be ligated, such as an external tissue of inverted LAA. In some embodiments, when the inner diameter of the loop 41 is unidirectionally contracted until the snare area of the loop 41 reaches a preset snare area, the loop 41 may be firmly sleeved on the target tissue, and thus the ligation operation of the target tissue may be completed. That is, when the loop 41 may be contracted to reach the preset snare area, the loop 41 may be formed a loop stopping in the reverse direction, which may be determined that the ligation operation of the target tissue is completed. The loop 41 may be pre-mounted in a receiving channel of the snare 3. The loop 41 may be moved out of the snare 3 through/from the slit 301 to outside of the receiving channel of the snare 3, and then the loop 41 may be disposed in space which may be enclosed by the inner sidewall of the snare 3 and outside the receiving channel. The space may be also referred to as an inner side enclosed by the snare 3.

When the second handle 2 is separated from the first handle 1 and the second handle 2 may be moved/pulled rearward, i.e., pulled away/move in a direction away from the first handle 1 or away from the snare 3, the ligature 4 may be driven to remove/pull rearward, such that the loop 41 may be driven to removed out/pulled out of the snare 3 from the slit to an inner side enclosed by the snare 3 and contract in the forward direction.

A ligature-cutting assembly 12 may be disposed in the second handle 2. When the second handle 2 is separated from the first handle 1 and pulled rearward until a preset action force, such as a pulling force, is reached, the ligature-cutting assembly 12 may be driven to move and cut the ligature 4.

An operation process of the automatic ligature-cutting ligation device 100 provided by some embodiments of the present disclosure may be described as follows. An end of the catheter assembly 12 connected to the snare 3 may be delivered into a heart through an outer sheath 200, and the handle assembly 11 11 may be disposed outside of the human body. Subsequently, the snare 3 may be sleeved on the external tissue of inverted LAA inside a cardiac cavity. Subsequently, the second handle 2 may be rotated to be separated from the first handle 1, and the second handle 2 may be pulled rearward in the direction away from the snare 3 (i.e., in a direction away from the human body), such that the loop 41 may be pulled out of the snare 3 from the slit, i.e., from the receiving channel, to the inner side enclosed by the snare 3, and the loop 41 may be contracted on a tissue to be ligated. As an action force, such as a pulling force, applied on the second handle 2 is increased, the inner diameter of the loop 41 may continue to contract towards the sleeved target tissue until the loop 41 may be firmly sleeved on the target tissue. The action force applied on the second handle 2 may also reach the preset action force when the tissue ligation is completed. At this time, the ligature-cutting assembly 12 may move under the pulling force and cut the ligature 4, such that an automatic ligature-cutting operation after a ligation operation may be completed.

Therefore, in the automatic ligature-cutting ligation device 100 provided by some embodiments of the present disclosure, the ligation operation may be implemented by separating the second handle 2 from the first handle 1, and the automatic ligature-cutting operation may be implemented when the pulling force applied on the second handle reaches the preset pulling force during the operation process, such that it is not necessary to rely on the visual judgment and hand feeling of the operating doctor. Therefore, it may be possible to reduce the requirement for the visual judgment and hand feeling when the doctor performs the ligation operation, thereby improving the operation accuracy and convenience, and reducing the operation difficulty.

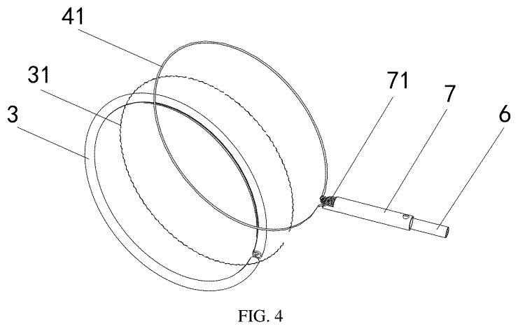

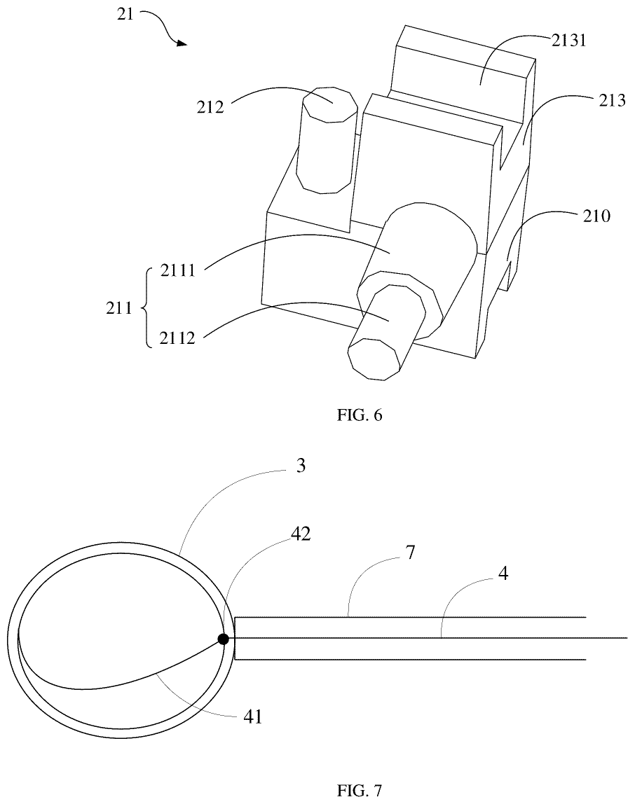

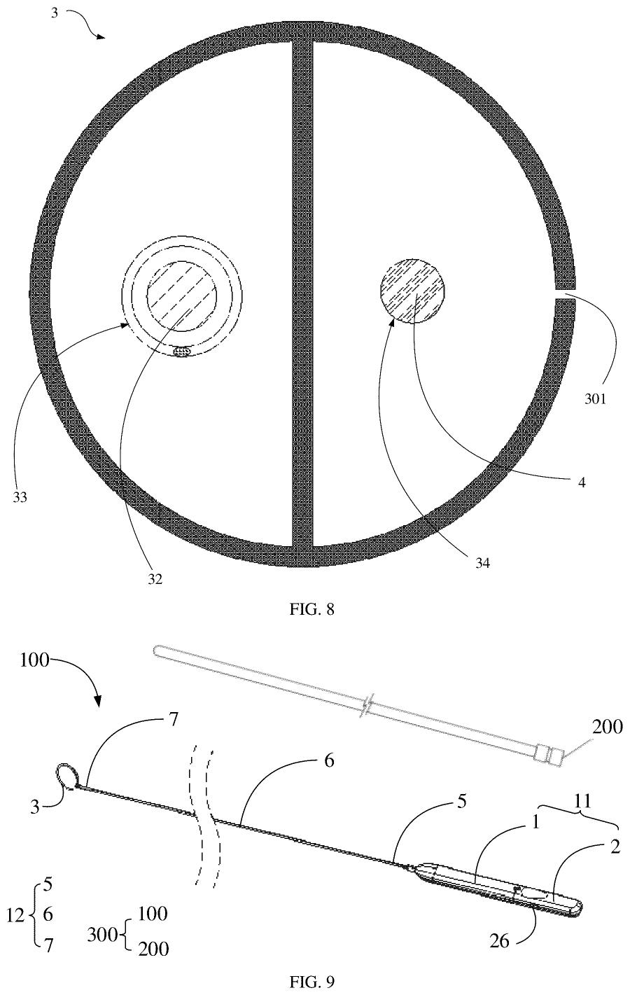

As shown in FIG. 4 and FIGS. 7-8, in some embodiments, the snare 3 may be a flexible ring with built-in dual channels. The dual channels may be disposed externally and internally, and the loop 41 may be embedded in an inner channel of the dual channels. In some embodiments, as shown in FIG. 8, the dual channels may include a first channel 33 and a second channel 34 arranged from outside to inside along a radial direction of the snare 3. The first channel 33 and the second channel 34 may be separated from each other. The first channel 33 may be an outer channel, and a deformable skeleton ring 32 may be disposed in the first channel 33 and configured to support the snare 3, such that it may be convenient for the subsequent positioning of the snare 3 on the target tissue. The second channel 34 may be an inner channel. The slit 301 may be defined on the inner surface of the snare 3 and in communication with the second channel 34. Therefore, the loop 41 may be pre-mounted or embedded in the second channel 34 through the slit 301. Alternatively, the loop 41 may be moved out of the snare 3 through/from the slit 301 to the outside of the second channel 34. The second channel 34 may be the aforementioned “receiving channel”.

The ligature 4 may pass through both ends of the catheter assembly 12 12. An end of the ligature 4 may pass through the first handle 1 and connected to the ligature-cutting assembly 13 disposed in the second handle 2. Another end of the ligature 4 may have a one-way knot 42 and form the loop 41 capable of contracting in the forward direction and stopping in the reverse direction. In some embodiments, the one-way knot 42 may be a knot in the related art. For example, a knotting method of the one-way knot 42 may be referred to a knotting method of a Roeder's knot. Of course, the knotting method of the one-way knot 42 may be not limited thereto, as long as the loop 41 may be contracted in the forward direction.

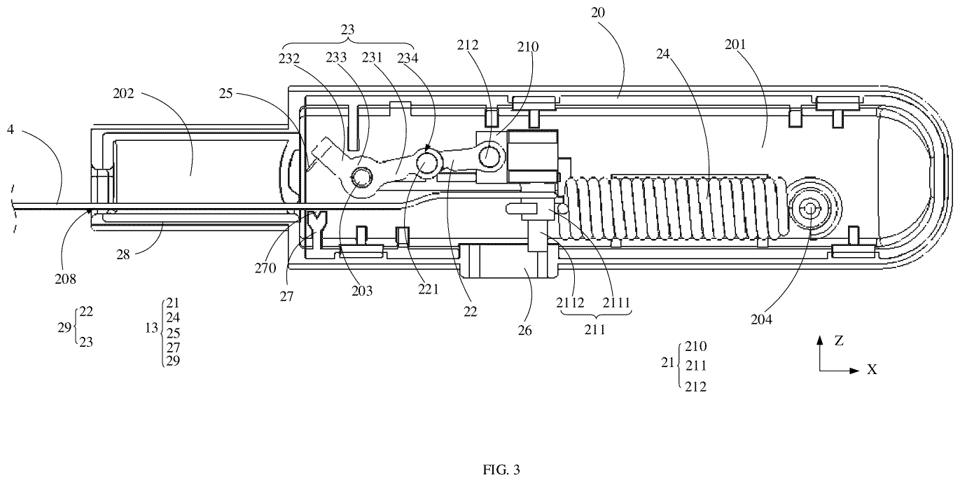

In some embodiments, as shown in FIGS. 2-3, the second handle 2 may include a grip portion 20 and an engagement portion 28 connected to each other. The grip portion 20 may be disposed at the rear end of the second handle 2. The grip portion 20 may be a hollow shell and have a first accommodating chamber 201 configured to accommodate the ligature-cutting assembly 13. The engagement portion 28 may be disposed at the front end of the second handle 2 (i.e., the end of the second handle 2 close to the snare 3 and disposed at an end of the grip portion 20 close to the first handle 1. In some embodiments, in a plane substantially perpendicular to an extending direction of the second handle 2, a projection of the engagement portion 28 may be located within a projection of the grip portion 20. The extending direction of the second handle 2 may be referred to a direction in which a length of the second handle 2 may be the longest, and may be substantially parallel to an X-axis direction as shown in FIG. 3. The engagement portion 28 may be arranged with a matching structure (such as a thread, a buckle, a clamping groove, etc.), and the matching structure may be configured to perform the detachable connection between the first handle 1 and the second handle 2. During assembly, the engagement portion 28 may be received in the first handle 1, and the detachable connection between the first handle 1 and the second handle 2 may be implemented through the matching structure. The engagement portion 28 may also be a hollow shell and have a second accommodating chamber 202. The second accommodating chamber 202 may be in communication with the first accommodating chamber 201, such that the ligature 4 may pass through the first accommodating chamber 201 and the second accommodating chamber 202. A through hole 208 may be defined on an end surface of the engagement portion 28 away from the first accommodating chamber 201. Therefore, an end of the ligature 4 may enter the second accommodating chamber 202 through the through hole 208, enter the first accommodating chamber 201, and be connected to the ligature-cutting assembly 13.

The ligature-cutting assembly 13 may be disposed in the second handle 2 and connected to the ligature 4. The ligature-cutting assembly 13 may be configured to cut the ligature 4 when the pulling force applied on the second handle 2 reaches the preset pulling force. When the second handle 2 moves in the direction away from the first handle 1, the ligature 4 may be subjected to a backward pulling force (i.e., a direction of the force is away from the first handle 1) applied by the ligature-cutting assembly 13 in the second handle 2, and the ligature 4 may react on the ligature-cutting assembly 13, such that it may be possible to enable the ligature-cutting assembly 13 to move forward, i.e., to enable the ligature-cutting assembly 13 to move in the direction close to the first handle 1. When the pulling force applied on the second handle 2 reaches the preset pulling force, the ligature-cutting assembly 13 may be driven to move to a preset ligature-cutting node, such that it may be possible to enable a blade of the ligature-cutting assembly 13 to collide with the ligature 4 to cut the ligature 4. In this way, by pulling the second handle 2 backward, it may be possible to complete the tissue ligation, and the ligature 4 may also be cut.

In some embodiments, the pulling force required for completing the ligation operation may be measured, the preset pulling force applied on the second handle 2 may be set according to the required pulling force, such that a ligature-cutting node of the ligature-cutting assembly 12 may be designed. In some embodiments, the pulling force required for completing the ligation may be generally about 30 N, and thus the preset pulling force may be designed to be 30 N. When the preset pulling force reaches 30 N, the ligature-cutting assembly 12 may be subjected to an action force of about 30 N, such as a pulling force of about 30 N. At this time, the ligature-cutting assembly 12 may be driven to move to the ligature-cutting node and implement the cutting operation, such that it may be possible to enable a blade of the ligature-cutting assembly 12 to collide with the ligature 4 to cut the ligature 4. In this way, by pulling the second handle 2 rearward, it may be possible to complete the tissue ligation, and the ligature 4 may also be cut, such that by adopting the cutting manner, during the operation process, it may be not necessary to rely on the visual judgment and hand feeling of the operating doctor, and thus it may be possible to reduce the errors or deviations caused by manual operation, thereby improving the accuracy of the ligation operation.

It should be noted that the preset pulling force of 30 N may be selected according to an actual application scenario in some embodiments of the present disclosure, and a specific value of the preset pulling force may be adaptively adjusted according to different situations. In some embodiments, the preset pulling force may be in a range from 20 N to 40 N, such as 20 N, 25 N, 28 N, 32 N, 35 N, 40 N, etc.

In some embodiments, the first handle 1 may be detachably connected to the second handle 2, such that the second handle 2 may be controlled to separate from the first handle 1 after the loop 41 is sleeved on the target tissue, thereby performing the ligation operation, so as to conveniently drive the ligation operation. In some embodiments, a rear end of the first handle 1 may be spirally interlocked with a front end of the second handle 2. When the second handle 2 rotates relative to the first handle 1, the second handle 2 may be capable of being separated from or engaged with the first handle 1. The wording “a front end” of a component may be referred to an end of the component close to the snare 3. The wording “a rear end” of the component may be referred to an end of the component away from the snare 3. Therefore, during the process of using the automatic ligature-cutting ligation device 100, the first handle 1 may be usually closer to the snare 3 than the second handle 2, and thus the first handle I may also be called as a front-section handle, and the second handle 2 may be called as a rear-section handle. In some embodiments, the first handle 1 may be detachably connected to the second handle 2 through a threaded structure.

In some embodiments, mutually matching buckle structures may be respectively arranged on an inner wall of the rear end of the first handle 1 and an inner wall of the front end of the second handle 2. By rotating the first handle 1 and the second handle 2 relative to each other, the first handle 1 and the second handle 2 may be relatively locked and fixed to implement the locking in a non-use state. In addition, the first handle 1 and the second handle 2 may also be unlocked from each other and pulled after unlocking to implement the quick operation during use. Therefore, only actions of rotation and pulling may be needed during use, and the operation may be simple and convenient. Of course, in other embodiments, the first handle 1 may also be detachably connected to the second handle 2 through other structures, such as a magnetic attraction structure, as long as the first handle 1 may be capable of being separated from or connected to the second handle 2.

As shown in FIG. 3, in some embodiments, the ligature-cutting assembly 12 may include a sliding member 21, a connecting rod member 29, an elastic member 24, and a ligature-cutting blade 25. The sliding member 21 may be slidably mounted in the second handle 2 and capable of sliding forward/rearward. That is, the sliding member 21 may be slidably mounted in the first accommodating chamber 201 and capable of sliding in a direction close to the engagement portion 28 or away from the engagement portion 28. In some embodiments, a sliding direction of the sliding member 21 may be substantially parallel to an axial direction of the second handle 2 i.e., the X-direction in FIG. 3. An end of the ligature 4 may be connected to the sliding member 21. In some embodiments, the ligature 4 may pass through the through hole 208, enter into the second accommodating chamber 202, enter the first accommodating chamber 201, and be connected to the sliding member 21. The connecting rod member 29 may be mounted in the second handle 2, and disposed at a side of the sliding member 21 close to the snare 3. An end of the connecting rod member 29 away from the snare 3 may be connected to the sliding member 21. The ligature-cutting blade 25 may be disposed at another end of the connecting rod member 29 close to the snare 3. The elastic member 24 may be disposed at the side of the sliding member 21 away from the snare 3. An end of the elastic member 24 away from the snare 3 may be connected to the inner wall of the second handle 2. Another end of the elastic member 24 close to the snare 3 may be connected to the sliding member 21. In other words, in a direction from the first handle 1 to the second handle 2, the ligature-cutting blade 25, the connecting rod member 29, the sliding member 21, and the elastic member 24 may be sequentially arranged in the first accommodating chamber 201.

When the second handle 2 is pulled rearward, i.e., pulled in a direction away from the snare 3 or away from the first handle 1, the ligature 4 may be configured to drive the sliding member 21 to slide forward, i.e., to drive the sliding member 21 to slide in a direction close to the first handle 1 or close to the snare 3, and the elastic member 24 may be driven to stretch in the direction close to the first handle 1 or close to the snare 3. The ligature 4 may be configured to simultaneously drive the connecting rod member 29 to rotate, so as to enable the ligature-cutting blade 25 to rotate toward the ligature 4. When the elastic member 24 is stretched to the preset pulling force, the ligature-cutting assembly 13 may move to the ligature-cutting node, such that the ligature-cutting blade 25 may rotate to contact the ligature 4 and cut the ligature 4. It should be understood that when the elastic member 24 is stretched to the preset pulling force, which should also be understood that the elastic member 24 is stretched to a preset stretching length.

In some embodiments, when the second handle 2 is pulled rearward 1, i.e., pulled in a direction away from the snare 3 or away from the first handle 1, the ligature 4 may exert/generate a forward pulling force on the sliding member 21, such that the sliding member 21 may be driven to move forward. In this way, the connecting rod member 29 may be driven to rotate through a forward movement of the sliding member 21, such that as the connecting rod member 29 rotates, the ligature-cutting blade 25 may be driven to rotate toward the ligature 4. Therefore, when the pulling force applied on the second handle 2 reaches the preset value, the ligature 4 may be cut by the ligature-cutting blade 25.

As shown in FIG. 5, FIG. 5 is a schematic structural view of a ligature-cutting assembly 12 mounted on the second handle in FIG. 1 according to another embodiment of the present disclosure. In some embodiments, the end of the elastic member 24 close to the snare 3 may be sleeved on the sliding member 21, and an end of the ligature 4 away from the snare 3 may be connected to the elastic member 24. When the second handle 2 may be pulled in a direction away from the snare 3 or the first handle 1, since the ligature 4 is connected to the elastic member 24 and the elastic member 24 is connected to the sliding member 21, the end of the ligature 4 away from the snare 3 may exert the forward pulling force on the elastic member 24. In this way, it may be possible to enable the ligature 4 to drive the elastic member 24 to stretch in the direction close to the snare 3, such that it may be possible to enable the elastic member 24 to push the sliding member 21 to slide in the direction close to the snare 3. Therefore, the connecting rod member 29 may be driven to rotate through the forward movement of the sliding member 21, such that as the connecting rod member 29 rotates, the ligature-cutting blade 25 may be driven to rotate toward the ligature 4. Therefore, when the pulling force applied on the second handle 2 reaches the preset value, the ligature-cutting assembly 13 may move to the ligature-cutting node, such that the ligature-cutting blade 25 may rotate to contact the ligature 4 and cut the ligature 4.

In some embodiments, the elastic member 24 may be a spiral spring, an elastic rope, or other structures with a rebound function, provided that the following conditions are satisfied. That is, when the automatic ligature-cutting ligation device 100 is in use, a stretching degree of the elastic member 24 may ensure that the pulling force applied on the second handle 2 may reach the preset pulling force, and the elastic member 24 is stretched to the preset pulling force when the pulling force applied on the second handle 2 reaches the preset value. When the automatic ligature-cutting ligation device 100 is not in use, the elastic member 24 may be capable of keeping the sliding member 21 in a reset state.

As shown in FIG. 3, in some embodiments, the connecting rod member 29 may include a first connecting rod 22 and a second connecting rod 23. An end of the first connecting rod 22 may be rotatably connected to the sliding member 21, and another end of the first connecting rod 22 may be rotatably connected to an end of the second connecting rod 23. A rod arm of the second connecting rod 23 may be rotatably connected to the inner wall of the second handle 2. The ligature-cutting blade 25 may be disposed at another end of the second connecting rod 23, and at this time, the elastic member 24 may be stretched. When the automatic ligature-cutting ligation device 100 is in use, the forward movement of the sliding member 21 may push the first connecting rod 22 and the second connecting rod 23 to rotate, so as to drive the ligature-cutting blade 25 to rotate toward the ligature 4. When the automatic ligature-cutting ligation device 100 is not in use, the elastic member 24 may be capable of keeping the sliding member 21 in the reset state, so as to keep the ligature-cutting blade 25 away from the ligature 4, thereby prevent accidental cutting. The connecting rod member 29, the elastic member 24, etc., may be arranged on the ligature-cutting assembly 13 provided in some embodiments of the present disclosure, such that it may be possible to reduce the probability of mis-cutting the ligature, thereby improving the operation accuracy.

As shown in FIG. 3, the first connecting rod 22 may include a connecting shaft 221 disposed at an end of the first connecting rod 22 close to the second connecting rod 23. The second connecting rod 23 may be rotatably connected to the connecting shaft 221. The second connecting rod 23 may include a first rod portion 231 and a second rod portion 232 connected to each other. The first rod portion 231 may be connected to the first connecting rod 22. The second rod portion 232 may be connected to the ligature-cutting blade 25. The first rod portion 231 and the second rod portion 232 may be arranged at an angle. The second connecting rod 23 may further include a rotational/rotary mounting portion 233 connected to the first rod portion 231 and the second rod portion 232. An end of the second rod portion 232 away from the rotational mounting portion 233 may be rotatably/pivotally connected to the connecting shaft 221. The rotational mounting portion 233 may be rotatably connected to the inner wall of the second handle 2. The ligature-cutting blade 25 may be disposed at the end of the second rod portion 232 away from the rotational mounting portion 233. In some embodiments, the rotational mounting portion 233 may also be referred to as a rod arm of the second connecting rod 23. The second connecting rod 23 may further include a connecting hole 234 defined at an end of the second rod portion 232 close to the first connecting rod 22. The connecting shaft 221 may be rotatably connected to the connecting hole 234, such that the first connecting rod 22 and the second connecting rod 23 may form a linkage structure. In some embodiments, a first limit shaft 203 and a second limit shaft 204 may be arranged at intervals on the inner wall of the second handle 2. The rotational mounting portion 233 may be sleeved on and rotatably connected to the first limit shaft 203. The end of the elastic member 24 away from the snare 3 may be sleeved on the second limit shaft 204. In some embodiments, the first limit shaft 203 and the second limit shaft 204 may be arrange at intervals substantially along the axial direction of the second handle 2, such that the elastic member 24 and the sliding member 21 may move linearly along the axial direction of the second handle 2.

As shown in FIG. 3, in some embodiments, an opening 26 may be defined on the second handle 2. The opening 26 may penetrate a sidewall 209 of the second handle 2. The opening 26 may be strip-shaped. In the plane substantially parallel to the axial direction of the second handle 2, at least a part of the projection of the sliding member 21 may be located within the opening 26. An extension portion 211 may be arranged on the sliding member 21. The extension portion 211 may pass through the opening 26 and may protrude beyond the second handle. Another end of the elastic member 24 close to the snare 3 may be connected to the extension portion 211.

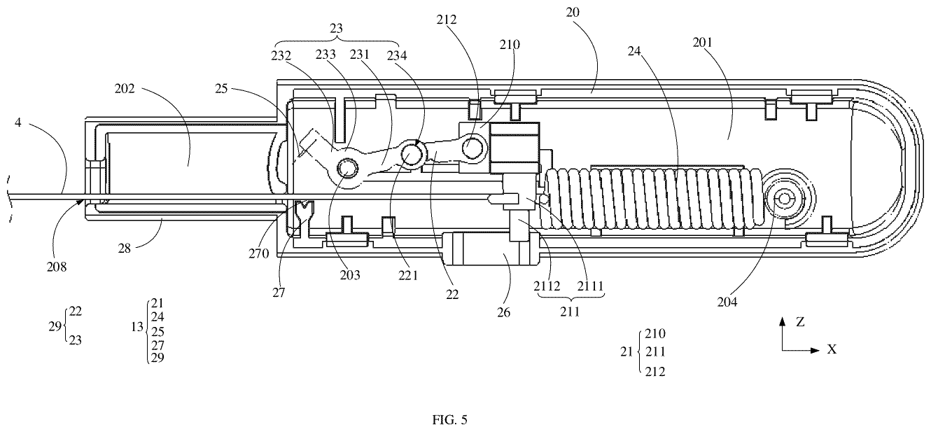

As shown in FIG. 6, FIG. 6 is a schematic structural view of a sliding member according to some embodiments of the present disclosure. In some embodiments, the sliding member 21 may further include a base portion 210. The base portion 210 may be disposed at a side of the extension portion 211 away from the opening 26. The base portion 210 may be accommodated in the first accommodating chamber 201. The extension portion 211 may include a first extension portion 2111 and a second extension portion 2112. The first extension portion 2111 may be disposed at a side of the base portion 210 close to the opening 26. The second extension portion 2112 may extend from the first extension portion 2111 in a direction away from the base portion 210. In a plane substantially perpendicular to a plane where the base portion 210 may be located, that is, in the plane substantially parallel to the axial direction of the second handle 2, a projection of the second extension portion 2112 may be located within a projection of the first extension portion 2111. The end of the elastic member 24 close to the snare 3 may be sleeved on the first extension portion 2111. The end of the ligature 4 away from the snare 3 may be connected to at least one of the first extension portion 2111 of the sliding member 21 and the elastic member 24.

In some embodiments, in a case where the end of the elastic member 24 away from the snare 3 is connected to the sliding member 21, such as the first extension portion 2111, when the second handle 2 moves in the direction away from the first handle 1, the ligature 4 may exert the forward pulling force on the sliding member 21, such as the first extension portion 2111, such that it may be possible to enable the ligature 4 to drive the sliding member 21 to slide in the direction close to the snare 3, thereby driving the elastic member 24 to stretch in the direction close to the snare 3. In some embodiments, in a case where the end of the ligature 4 away from the snare 3 is connected to the elastic member 24, when the second handle 2 moves in the direction away from the first handle 1, the ligature 4 may exert the forward pulling force on the elastic member 24, such that it may be possible to enable the ligature 4 to drive the elastic member 24 to stretch in the direction close to the snare 3, so as to enable the elastic member 24 to push the sliding member 21 to slide in the direction close to the snare 3. In some embodiments, in a case where the end of the ligature 4 away from the snare 3 is connected to both the sliding member 21 (such as the first extension portion 2111) and the elastic member 24, when the second handle 2 moves in the direction away from the first handle 1, the ligature 4 may exert the forward pulling force on at least one of the sliding member 21 (such as the first extension portion 2111) and the elastic member 24, such that it may be possible to enable the ligature 4 to drive the sliding member 21 to slide in the direction close to the snare 3 and/or enable the ligature 4 to drive the elastic member 24 to stretch in the direction close to the snare 3. That is to say, the end of the ligature 4 away from the snare 3 may be connected to either the sliding member 21 (such as the first extension portion 2111) or the elastic member 24, or may be connected to both the extension portion 211 (such as the first extension portion 2111) and the elastic member 24. Since the elastic member 24 may be sleeved on the sliding member 21 (such as the first extension portion 2111), regardless of which of the above connection methods is employed, it can be realized that the ligature 4 may drive the elastic member 24 to stretch in the direction close to the snare 3 until the elastic member 24 reaches the preset stretching length, such that the ligature-cutting assembly 13 reaches the ligature-cutting node. Therefore, in a case where the elastic member 24 is stretched to the preset pulling force or the preset stretching length, the ligature-cutting assembly 13 may be driven move to the ligature-cutting node and cut the ligature 4.

In some embodiments, the sliding member 21 may further include a fixing pillar 212 disposed on a top surface of the base portion 210. An end of the first connecting rod 22 away from the second connecting rod 23 may be sleeved on and rotatably connected to the fixing pillar 212. The sliding member 21 may further include a protrusion 213 spaced apart from the fixing pillar 212. A groove 2131 may be defined on a top surface of the protrusion 213. A contour of the groove 2131 may substantially match a shape of a mating component arranged on the inner wall of the second handle 2. In some embodiments, the second handle 2 may include a first housing and a second housing. The first housing and the second housing may be snap-fitted to define the first accommodating chamber 201 and the second accommodating chamber 202. The first housing may be configured to accommodate the ligature-cutting assembly 13. A guiding member may be disposed on an inner wall of the second housing and opposite to the ligature-cutting assembly 13. The sliding member 21 may be slidably connected to the guiding member via the groove 2131. The sliding member 21 may be capable of sliding on the guiding member in a direction substantially parallel to the axial direction of the second handle 2.

In some embodiments, a length direction of the opening 26 may be substantially parallel to a sliding direction of the sliding member 21. The extension portion 211 may be disposed in the opening 26, such that it may be possible to limit a sliding path of extension portion 211, thereby limiting a sliding path of the elastic member 24. In this way, on one hand, a sliding stroke of the sliding member 21 may be limited, such that it may be possible to limit the maximum pulling force achievable by pulling the second handle 2 backward, thereby ensuring that excessive force does not occur. On the other hand, the extension portion 211 may protrude beyond the second handle 2, such that it may be possible to allow the operating doctor to visually observe a change in the sliding stroke externally, thereby enabling indirect control of the pulling force magnitude during the ligation process and helping to prevent an operational error. It may be understood that at least a part of the extension portion 211, for example, at least a part of the second extension portion 2112, may be disposed in the opening 26 without protruding beyond the side wall 209 of the second handle 2. In other words, an end of the extension portion 211, for example, an end of the second extension portion 2112, may protrude out of the opening 26 or may not protrude out of the opening 26. The end of the extension portion 211, for example, the end of the second extension portion 2112, may be substantially flush with the side wall 209 of the second handle 2. Alternatively, the end of the extension portion 211, for example, the end of the second extension portion 2112, may be slightly recessed below the side wall 209 of the second handle 2. Alternatively, the end of the extension portion 211, for example, the end of the second extension portion 2112, may protrude beyond the side wall 209 of the second handle 2. Any configuration may be acceptable as long as the extension portion 211 may remain disposed in the opening 26 to limit the sliding path of the elastic member 24.

As shown in FIG. 3, in some embodiments, a ligature-cutting base 27 may be arranged in the second handle 2 and disposed at a side of the grip portion 20 close to the snare 3, such that it may be possible to ensure that the ligature 4 may be rapidly cut by the ligature-cutting blade 25 under the preset pulling force. The ligature-cutting blade 25 may be opposite to the ligature-cutting base 27. The ligature 4 may be placed on the ligature-cutting base 27. A notch/groove 270 may be defined on the ligature-cutting base 27 and opposite to the ligature-cutting blade 25. When it is necessary to cut the ligature, the ligature-cutting blade 25 may be capable of rotating to abut against the notch 270, and the ligature 4 may be subjected to greater tension at the notch 270, such that the ligature 4 may be rapidly cut. As shown in FIG. 3, the notch 270 may be a V-shaped opening/groove. Of course, in other embodiments, the notch 270 may also be an opening of other shapes, such as a U-shaped opening, an arc-shaped opening, a serrated opening, etc., as long as it can enable the ligature 4 be subjected to the greater tension at the notch 270. In some embodiments, when the ligature-cutting blade 25 rotates to contact the ligature-cutting base 27, for example, when the ligature-cutting blade 25 rotates to contact the notch 270, a node where the ligature 4 contacts the notch 270 may be defined as the ligature-cutting node of the ligature-cutting assembly 13.

In some embodiments, the ligature-cutting blade 25 may be a hard ceramic blade, and the blade may be hard and sharp, thereby enabling rapid completion of the cutting process. In this way, it may be possible to reduce the risk of over-tightening the loop 41 and scratching cardiac tissue.

As shown in FIGS. 1, 2, and 4, in some embodiments, the catheter assembly 12 may include a heat-shrinkable sleeve 5, a flexible catheter 6, and a connecting tube 7. An end of the flexible catheter 6 away from the snare 3 may be connected to the front end of the first handle 1 close to the snare 3. A heat-shrinkable sleeve 5 may be covered on a connection part between the flexible catheter 6 and the front end of the first handle I close to the snare 3. An end of the connecting tube 7 may be connected to another end of the flexible catheter 6 close to the snare 3. Another end of the connecting tube 7 may be connected to the snare 3. That is, in the direction from the first handle I to the second handle 2, the snare 3, the connecting tube 7, the flexible catheter 6, and first handle 1 may be sequentially connected.

In some embodiments, the flexible catheter 6 may be made of flexible tubing material, which may offer excellent resistance to kinking, fracture, and plastic deformation, such that the flexible catheter 6 may be suitable for various bending scenarios, thereby significantly improving clinical efficiency. The connecting tube 7 may be a metal component, so as to provide sufficient strength at a front end of the handle assembly 11, thereby ensuring optimal retraction of the loop 41.

In some embodiments, the snare 3 may be designed with different diameter specifications according to different requirements. The whole snare 3 may be wrapped by a soft thermoplastic polyurethane (TPU) material, such it may be possible to protect a myocardial tissue and reduce accidental injury. A bone positioning component or a skeleton (i.e., the skeleton ring 32) inside the snare 3 may be made of a nitinol memory alloy wire, so as to meet the usage environment or a channel with a narrow or curved condition.

In some embodiments, the snare 3 may be a flexible ring with built-in dual channels. The dual channels may be disposed externally and internally, and the loop 41 may be embedded in an inner channel of the dual channels.

As shown in FIG. 4, in some embodiments, a radiopaque tungsten wire 31 may be embedded in an outer channel of the snare 3, and a radiopaque coil spring 71 may be disposed in the connection tube 7, so as to enhance imaging clarity and prevent an operational error. In this way, it may be possible to enable a position of the snare 3 to be clearly displayed on an imaging module via the radiopaque tungsten wire 31 and the radiopaque coil spring 71, thereby improving operability.

As shown in FIG. 9, a ligation system 300 may further be provided in some embodiments of the present disclosure. A ligation system 300 may include the automatic ligature-cutting ligation device 100 according to any of the above embodiments and an outer sheath 200. The outer sheath 200 may be configured to deliver an end of the automatic ligature-cutting ligation device 100 where the snare 3 is disposed into a heart. Under an external force, the end of the automatic ligature-cutting ligation device 100 where the snare 3 is disposed delivered into the heart may perform ligation on the LAA, a specific process of which may be referred to the relevant content in the above embodiments. It may be understood that the ligation system 300 provided in the embodiments of the present disclosure may have the same technical effects as the automatic ligature-cutting ligation device 100 in the embodiments of the present disclosure, which will not be repeated herein.

According to a first aspect, some embodiments of the present disclosure provide an automatic ligature-cutting ligation device. The automatic ligature-cutting ligation device includes: a handle assembly, including a first handle and a second handle detachably connected to each other; a catheter assembly, an end of the catheter assembly being connected to the first handle; a snare, disposed at another end of the catheter assembly, where a slit is defined in an inner surface of the snare; a ligature, penetrating the first handle and the catheter assembly, where an end of the ligature is connected to the second handle, another end of the ligature has a loop capable of contracting in a forward direction and stopping in a reverse direction, and the loop is pre-mounted in the snare; and a ligature-cutting assembly, disposed in the second handle; where in a case where the second handle is separated from the first handle and pulled out in a direction away from the first handle, the ligature is driven to pull out in the direction away from the first handle, and the loop is driven to pull out of the snare from the slit to an inner side enclosed by the snare and contract in the forward direction; and in a case where the second handle is separated from the first handle and pulled out in the direction away from the first handle until a preset pulling force is reached, the ligature-cutting assembly is driven to move and cut the ligature.

In some embodiments, an end of the first handle close to the second handle is spirally interlocked with an end of the second handle close to the first handle, and in a case where the second handle rotates relative to the first handle, the second handle is capable of being separated from or engaged with the first handle.

In some embodiments, the ligature-cutting assembly includes: a sliding member, slidably mounted in the second handle and capable of sliding in a direction close to the first handle or away from the first handle, where an end of the ligature is connected to the sliding member; a connecting rod member, mounted in the second handle, where an end of the connecting rod member is connected to the sliding member; an elastic member, an end of the elastic member being connected to an inner side of the second handle, and another end of the elastic member being connected to the sliding member; and a ligature-cutting blade, connected to another end of the connecting rod member; where in a case where the second handle is pulled out in the direction away from the first handle, the ligature is configured to drive the sliding member to slide in the direction close to the first handle, the elastic member is driven to stretch, and the connecting rod member is simultaneously driven to rotate, such that the ligature-cutting blade is driven to rotate towards the ligature; and in a case where the elastic member is stretched to the preset pulling force, the ligature is cut by the ligature-cutting blade.

In some embodiments, the connecting rod member includes a first connecting rod and a second connecting rod; an end of the first connecting rod is rotatably connected to the sliding member, and another end of the first connecting rod is rotatably connected to an end of the second connecting rod; and a rod arm of the second connecting rod is rotatably connected to an inner wall of the second handle, and the ligature-cutting blade is disposed at another end of the second connecting rod.

In some embodiments, an opening is defined on the second handle, the sliding member is arranged with an extension portion, and the extension portion is connected to another end of the elastic member. The extension portion penetrates out of the opening and protrudes beyond a side wall of the second handle; or at least a part of the extension portion is disposed in the opening without protruding beyond a side wall of the second handle.

In some embodiments, the elastic member is an elastic member with a rebound function, and the elastic member includes one of a spiral spring and an elastic pull rope.

In some embodiments, a ligature-cutting base is disposed in the second handle, the ligature-cutting blade is opposite to the ligature-cutting base, and the ligature is placed on the ligature-cutting base; and a notch opposite to the ligature-cutting blade is defined on the ligature-cutting base.

In some embodiments, the catheter assembly includes a heat-shrinkable sleeve, a flexible catheter, and a connecting tube; an end of the flexible catheter is connected to an end of the first handle away from the second handle; the heat-shrinkable sleeve is covered on a connection part between the flexible catheter and the end of the first handle away from the second handle; and an end of the connecting tube is connected to another end of the flexible catheter, and another end of the connecting tube is connected to the snare.

In some embodiments, the snare includes a first channel and a second channel separated apart from each other, a skeleton ring is disposed in the first channel, the slit is defined on the second channel, and the loop is disposed in the second channel; and at least one of the skeleton ring and the loop includes a shape memory material, and the shape memory material includes a nitinol memory alloy wire.

In some embodiments, a radiopaque tungsten wire is embedded in the snare, and a radiopaque coil spring is disposed in the connecting tube.

According to a second aspect, some embodiments of the present disclosure provide an automatic ligature-cutting ligation device. The automatic ligature-cutting ligation device includes: a first handle; a second handle, detachably connected to the first handle; a catheter assembly, an end of the catheter assembly being connected to the first handle; a snare, disposed at another end of the catheter assembly, where a slit is defined in an inner surface of the snare; a ligature-cutting assembly, disposed in the second handle; and a ligature, penetrating the first handle and the catheter assembly, where an end of the ligature close to the snare is configured to form a unidirectional contraction loop, the loop is disposed in the snare, an end of the ligature away from the snare is connected to the ligature-cutting assembly; where in a case where the second handle is configured to move away from the first handle, the loop is driven to move out of the snare from the slit and contract unidirectionally; and in a case where the loop contracts unidirectionally until a snare area of the loop reaches a preset snare area, the ligature is configured to drive the ligature-cutting assembly to move towards the ligature until the ligature-cutting assembly reaches a ligature-cutting node and cuts the ligature.

In some embodiments, the snare includes a first channel and a second channel separated from each other, the first channel and the second channel are arranged along a direction from outside of the snare to inside of the snare, the slit is defined on the second channel, and the loop is embedded in the second channel.

In some embodiments, the second handle includes a grip portion and an engagement portion, the engagement portion is disposed at an end of the grip portion close to the first handle and is detachably connected to the first handle; and in a plane perpendicular to an axial direction of the second handle, a projection of the engagement portion is located within a projection of the handle portion.

In some embodiments, a first accommodating chamber is defined on the grip portion, a second accommodating chamber is defined on the engagement portion, the second accommodating chamber is in communication with the first accommodating chamber; a through hole is defined on an end face of the engagement portion away from the first accommodating chamber, and the ligature-cutting assembly is accommodated in the first accommodating chamber; and the end of the ligature away from the snare is configured to sequentially pass through the through hole and the second accommodating chamber, enter the first accommodating chamber, and be connected to the ligature-cutting assembly.

In some embodiments, the ligature-cutting assembly includes: a sliding member, slidably mounted in the first accommodating chamber, where the sliding member is configured to slide in the first accommodating chamber along a direction close to or away from the engagement portion; a connecting rod member, mounted in the first accommodating chamber, where the connecting rod member is disposed at a side of the sliding member facing the engagement portion and connected to the sliding member; an elastic member, mounted in the first accommodating chamber, where the elastic member is disposed at a side of the sliding member away from the engagement portion and connected to the sliding member; and a ligature-cutting blade, connected to an end of the connecting rod member close to the engagement portion. In a case where the second handle moves away from the first handle, the elastic member is driven to be stretched in a direction close to the engagement portion by at least one of the ligature and the sliding member, and the connecting rod member is driven to rotate in a direction close to the ligature until the ligature-cutting blade reaches the ligature-cutting node and cuts the ligature.

In some embodiments, the connecting rod member includes: a first connecting rod, an end of the first connecting rod away from the engagement portion being rotatably connected to the sliding member; and a second connecting rod, an end of the second connecting rod away from the engagement portion being rotatably connected to an end of the first connecting rod close to the engagement portion, where the ligature-cutting blade is disposed at an end of the second connecting rod close to the engagement portion.

In some embodiments, an end of the elastic member close to the snare is sleeved on the sliding member, and the end of the ligature away from the snare is connected to at least one of the elastic member and the sliding member; and in a case where the elastic member is stretched to a preset pulling force, the ligature-cutting assembly may be driven move to the ligature-cutting node and cuts the ligature.

In some embodiments, in a case where the end of the ligature away from the snare is connected to the elastic member, when the second handle moves in the direction away from the first handle, the ligature may exert the forward pulling force on the elastic member, such that it may be possible to enable the ligature to drive the elastic member to stretch in the direction close to the snare, so as to enable the elastic member to push the sliding member to slide in the direction close to the snare. Therefore, in a case where the elastic member is stretched to a preset pulling force or a preset stretching length, the ligature-cutting assembly may be driven move to the ligature-cutting node and cut the ligature. In some embodiments, in a case where a case where the end of the ligature away from the snare is connected to the sliding member, when the second handle moves in the direction away from the first handle, the ligature may exert the forward pulling force on the first extension portion, such that it may be possible to enable the ligature to drive the sliding member to slide in the direction close to the snare, thereby driving the elastic member to stretch in the direction close to the snare. Therefore, in a case where the elastic member is stretched to a preset pulling force or a preset stretching length, the ligature-cutting assembly may be driven move to the ligature-cutting node and cut the ligature. In some embodiments, in a case where the end of the ligature away from the snare is connected to both the sliding member and the elastic member, when the second handle moves in the direction away from the first handle, the ligature may exert the forward pulling force on at least one of the sliding member and the elastic member, such that it may be possible to enable the ligature to drive the sliding member to slide in the direction close to the snare and/or enable the ligature to drive the elastic member to stretch in the direction close to the snare. Therefore, in a case where the elastic member is stretched to a preset pulling force or a preset stretching length, the ligature-cutting assembly may be driven move to the ligature-cutting node and cut the ligature.

In some embodiments, an opening is defined on the second handle, the sliding member is arranged with an extension portion, and the extension portion penetrates out of the opening and protrudes beyond a side wall of the second handle; or at least a part of the extension portion is disposed in the opening without protruding beyond a side wall of the second handle; the sliding member includes a base portion, the base portion is disposed at a side of the extension portion away from the opening and is accommodated in the second handle; and an end of the elastic member close to the snare is sleeved on the extension portion, and the end of the ligature away from the snare is connected to at least one of the extension portion and the elastic member.

In some embodiments, the extension portion includes a first extension portion and a second extension portion, the first extension portion is disposed at a side of the base portion close to the opening, and the second extension portion extends from the first extension portion in a direction away from the base portion; and the end of the elastic member close to the snare is sleeved on the first extension portion, and the end of the ligature away from the snare is connected to at least one of the first extension portion and the elastic member.

According to a third aspect, an automatic ligature-cutting ligation device may be provided in some embodiments of the present disclosure. The automatic ligature-cutting ligation device may include: a first handle; a second handle, detachably connected to the first handle; a catheter assembly, an end of the catheter assembly being connected to the first handle; a snare, disposed at another end of the catheter assembly, where a slit may be defined in an inner surface of the snare; a sliding member, slidably mounted in the second handle, where the sliding member may be configured to slide in the second handle in a direction close to or away from the snare; and an elastic member, mounted in the second handle, where the elastic member may be disposed at a side of the sliding member away from the snare and connected to the sliding member; a ligature, penetrating the first handle and the catheter assembly, where an end of the ligature close to the snare may be configured to form a unidirectional contraction loop, the loop may be disposed in the snare. An end of the ligature away from the snare may be connected to at least one of the sliding member and the elastic member. In a case where the second handle is configured to move away from the first handle, the loop may be driven to move out of the snare from the slit and contract unidirectionally. In a case where the loop contracts unidirectionally until a snare area of the loop reaches a preset snare area, the ligature may be configured to drive the sliding member to move/slide in the direction close to the snare and/or drive the elastic member to stretch in the direction close to the snare until the elastic member may be stretched to a preset pulling force, such that the ligature may be cut.

In some embodiments, an opening is defined on the second handle. The extension portion penetrates out of the opening and protrudes beyond a side wall of the second handle; or at least a part of the extension portion is disposed in the opening without protruding beyond a side wall of the second handle.

In some embodiments, the sliding member may include a base portion. The base portion may be disposed at a side of the extension portion away from the opening and may be accommodated in the second handle. The extension portion may be connected to the end of the elastic member close to the snare, and the end of the ligature away from the snare may be connected to at least one of the extension portion and the elastic member.

In some embodiments, the extension portion may include a first extension portion and a second extension portion. The first extension portion may be disposed at a side of the base portion close to the opening. The second extension portion may extend from the first extension portion in a direction away from the base portion. The end of the elastic member close to the snare may be sleeved on the first extension portion. The end of the ligature away from the snare may be connected to at least one of the first extension portion and the elastic member.

In some embodiments, the automatic ligature-cutting ligation device may further include a first connecting rod, a second connecting rod, and a ligature-cutting blade. An end of the first connecting rod may be rotatably connected to the sliding member. Another end of the first connecting rod may be rotatably connected to an end of the second connecting rod. A rod arm of the second connecting rod may be rotatably connected to an inner wall of the second handle. The ligature-cutting blade may be disposed at another end of the second connecting rod.

In some embodiments, the sliding member may further include a fixing pillar disposed on a top surface of the base portion. The first connecting rod may be sleeved on the fixing pillar and rotatably connected to the fixing pillar.

In some embodiments, a first limiting shaft and a second limiting shaft may be arranged at intervals in the second handle. The second connecting rod may be sleeved on the first limiting shaft and rotatably connected to the first limiting shaft. The end of the elastic member away from the snare may be sleeved on the second limiting shaft.

According to a fourth aspect, some embodiments of the present disclosure provide a ligation system. The ligation system includes an automatic ligature-cutting ligation device, including: a handle assembly, including a first handle and a second handle detachably connected to each other; a catheter assembly, an end of the catheter assembly being connected to the first handle; a snare, disposed at another end of the catheter assembly, where a slit is defined in an inner surface of the snare; a ligature, penetrating the first handle and the catheter assembly, where an end of the ligature is connected to the second handle, another end of the ligature has a loop capable of contracting in a forward direction and stopping in a reverse direction, and the loop is pre-mounted in the snare; and a ligature-cutting assembly, disposed in the second handle; and an outer sheath, at least a part of the automatic ligature-cutting ligation device passing through the outer sheath, and the outer sheath being configured to deliver an end of the automatic ligature-cutting ligation device where the snare is disposed into a heart; where in a case where the second handle is separated from the first handle and pulled away from the first handle, the ligature is driven to be moved away from the first handle, and the loop is driven to be pulled out of the snare from the slit to an inner side enclosed by the snare and unidirectionally contract on the target tissue; and in a case where the second handle is separated from the first handle and pulled away from the first handle until a preset pulling force is reached, the ligature-cutting assembly is driven to move and cut the ligature.

An automatic ligature-cutting ligation device may be provided in some embodiments of the present disclosure. The automatic ligature-cutting ligation device may include a first handle, a second handle, a catheter assembly, a snare, a ligature, and a ligature-cutting assembly. An end of the catheter assembly may be connected to the first handle, and the snare may be disposed at another end of the catheter assembly. A slit may be defined in an inner surface of the snare. The ligature may penetrate the first handle and the catheter assembly. An end of the ligature may be connected to the second handle, and another end of the ligature may be a loop capable of contracting in a forward direction and stopping in a reverse direction. The loop may be pre-mounted in the snare. The ligature-cutting assembly may be disposed in the second handle. When the second handle is separated from the first handle and pulled away from the first handle, the ligature may be driven to be moved away from the first handle, and the loop is driven to be pulled out of the snare from the slit to an inner side enclosed by the snare and unidirectionally contract. When the second handle is separated from the first handle and pulled away from the first handle until a preset pulling force is reached, the ligature-cutting assembly may be driven to move and cut the ligature. That is, when the ligature is pulled away from the first handle until the preset pulling force may be reached, the ligature-cutting assembly may be driven to move to contact the ligature and automatically cut the ligature. In this way, in the automatic ligature-cutting ligation device provided in the embodiments of the present disclosure, by pulling the second handle away from the first handle, the loop may be driven to contract in the forward direction, thereby performing the ligation operation. On the one hand, the ligature-cutting assembly may be driven to move to contact the ligature and automatically cut the ligature. Therefore, during the above operation process, the ligature-cutting assembly may automatically cut the ligature at a ligature-cutting node, such that it is not necessary to rely on the visual judgment and hand feeling of the operating doctor, thereby reducing the occurrence of manual operation errors or deviations.

As described above, this is the specific embodiments of the present disclosure, but the protection scope of the present disclosure is not limited thereto. Any person skilled in the art may easily think of various equivalent modifications or substitutions within the technical scope disclosed in the present disclosure, and these modifications or substitutions should be covered within the protection scope of the present disclosure. Therefore, the protection scope of the present disclosure should be subject to the protection scope of the claims.

1. An automatic ligature-cutting ligation device, comprising:
a handle assembly, comprising a first handle and a second handle detachably connected to each other;
a catheter assembly, an end of the catheter assembly being connected to the first handle;
a snare, disposed at another end of the catheter assembly, wherein a slit is defined in an inner surface of the snare;
a ligature, penetrating the first handle and the catheter assembly, wherein an end of the ligature is connected to the second handle, another end of the ligature has a loop capable of contracting in a forward direction and stopping in a reverse direction, and the loop is pre-mounted in the snare; and
a ligature-cutting assembly, disposed in the second handle;
wherein in a case where the second handle is separated from the first handle and pulled out in a direction away from the first handle, the ligature is driven to pull out in the direction away from the first handle, and the loop is driven to pull out of the snare from the slit to an inner side enclosed by the snare and contract in the forward direction; and
in a case where the second handle is separated from the first handle and pulled out in the direction away from the first handle until a preset pulling force is reached, the ligature-cutting assembly is driven to move and cut the ligature.

2. The automatic ligature-cutting ligation device as claimed in claim 1, wherein an end of the first handle close to the second handle is spirally interlocked with an end of the second handle close to the first handle, and in a case where the second handle rotates relative to the first handle, the second handle is capable of being separated from or engaged with the first handle.

3. The automatic ligature-cutting ligation device as claimed in claim 1, wherein the ligature-cutting assembly comprises:
a sliding member, slidably mounted in the second handle and capable of sliding in a direction close to the first handle or away from the first handle, wherein an end of the ligature is connected to the sliding member;
a connecting rod member, mounted in the second handle, wherein an end of the connecting rod member is connected to the sliding member;
an elastic member, an end of the elastic member being connected to an inner side of the second handle, and another end of the elastic member being connected to the sliding member; and
a ligature-cutting blade, connected to another end of the connecting rod member;
wherein in a case where the second handle is pulled out in the direction away from the first handle, the ligature is configured to drive the sliding member to slide in the direction close to the first handle, the elastic member is driven to stretch, and the connecting rod member is simultaneously driven to rotate, such that the ligature-cutting blade is driven to rotate towards the ligature; and
in a case where the elastic member is stretched to the preset pulling force, the ligature is cut by the ligature-cutting blade.

4. The automatic ligature-cutting ligation device as claimed in claim 3, wherein the connecting rod member comprises a first connecting rod and a second connecting rod;
an end of the first connecting rod is rotatably connected to the sliding member, and another end of the first connecting rod is rotatably connected to an end of the second connecting rod; and
a rod arm of the second connecting rod is rotatably connected to an inner wall of the second handle, and the ligature-cutting blade is disposed at another end of the second connecting rod.

5. The automatic ligature-cutting ligation device as claimed in claim 3, wherein an opening is defined on the second handle, the sliding member is arranged with an extension portion, and the extension portion is connected to another end of the elastic member;
wherein the extension portion penetrates out of the opening and protrudes beyond a side wall of the second handle; or at least a part of the extension portion is disposed in the opening without protruding beyond a side wall of the second handle.

6. The automatic ligature-cutting ligation device as claimed in claim 3, wherein the elastic member is an elastic member with a rebound function, and the elastic member comprises one of a spiral spring and an elastic pull rope.

7. The automatic ligature-cutting ligation device as claimed in claim 3, wherein a ligature-cutting base is disposed in the second handle, the ligature-cutting blade is opposite to the ligature-cutting base, and the ligature is placed on the ligature-cutting base; and
a notch opposite to the ligature-cutting blade is defined on the ligature-cutting base.

8. The automatic ligature-cutting ligation device as claimed in claim 1, wherein the catheter assembly comprises a heat-shrinkable sleeve, a flexible catheter, and a connecting tube;
an end of the flexible catheter is connected to an end of the first handle away from the second handle;
the heat-shrinkable sleeve is covered on a connection part between the flexible catheter and the end of the first handle away from the second handle; and
an end of the connecting tube is connected to another end of the flexible catheter, and another end of the connecting tube is connected to the snare.

9. The automatic ligature-cutting ligation device as claimed in claim 1, wherein the snare comprises a first channel and a second channel separated apart from each other, a skeleton ring is disposed in the first channel, the slit is defined on the second channel, and the loop is disposed in the second channel; and
at least one of the skeleton ring and the loop comprises a shape memory material, and the shape memory material comprises a nitinol memory alloy wire.

10. The automatic ligature-cutting ligation device as claimed in claim 8, wherein a radiopaque tungsten wire is embedded in the snare, and a radiopaque coil spring is disposed in the connecting tube.

11. An automatic ligature-cutting ligation device, comprising:
a first handle;
a second handle, detachably connected to the first handle;
a catheter assembly, an end of the catheter assembly being connected to the first handle;
a snare, disposed at another end of the catheter assembly, wherein a slit is defined in an inner surface of the snare;
a ligature-cutting assembly, disposed in the second handle; and
a ligature, penetrating the first handle and the catheter assembly, wherein an end of the ligature close to the snare is configured to form a unidirectional contraction loop, the loop is disposed in the snare, an end of the ligature away from the snare is connected to the ligature-cutting assembly;
wherein in a case where the second handle is configured to move away from the first handle, the loop is driven to move out of the snare from the slit and contract unidirectionally; and
in a case where the loop contracts unidirectionally until a snare area of the loop reaches a preset snare area, the ligature is configured to drive the ligature-cutting assembly to move towards the ligature until the ligature-cutting assembly reaches a ligature-cutting node and cuts the ligature.

12. The automatic ligature-cutting ligation device as claimed in claim 11, wherein the snare comprises a first channel and a second channel separated from each other, the first channel and the second channel are arranged along a direction from outside of the snare to inside of the snare, the slit is defined on the second channel, and the loop is embedded in the second channel.

13. The automatic ligature-cutting ligation device as claimed in claim 11, wherein the second handle comprises a grip portion and an engagement portion, the engagement portion is disposed at an end of the grip portion close to the first handle and is detachably connected to the first handle; and
in a plane perpendicular to an axial direction of the second handle, a projection of the engagement portion is located within a projection of the handle portion.

14. The automatic ligature-cutting ligation device as claimed in claim 13, wherein a first accommodating chamber is defined on the grip portion, a second accommodating chamber is defined on the engagement portion, and the second accommodating chamber is in communication with the first accommodating chamber;
a through hole is defined on an end face of the engagement portion away from the first accommodating chamber, and the ligature-cutting assembly is accommodated in the first accommodating chamber; and
the end of the ligature away from the snare is configured to sequentially pass through the through hole and the second accommodating chamber, enter the first accommodating chamber, and be connected to the ligature-cutting assembly.

15. The automatic ligature-cutting ligation device as claimed in claim 14, wherein the ligature-cutting assembly comprises:
a sliding member, slidably mounted in the first accommodating chamber, wherein the sliding member is configured to slide in the first accommodating chamber along a direction close to or away from the engagement portion;
a connecting rod member, mounted in the first accommodating chamber, wherein the connecting rod member is disposed at a side of the sliding member facing the engagement portion and connected to the sliding member;
an elastic member, mounted in the first accommodating chamber, wherein the elastic member is disposed at a side of the sliding member away from the engagement portion and connected to the sliding member; and
a ligature-cutting blade, connected to an end of the connecting rod member close to the engagement portion;
wherein in a case where the second handle moves away from the first handle, the elastic member is driven to be stretched in a direction close to the engagement portion by at least one of the ligature and the sliding member, and the connecting rod member is driven to rotate in a direction close to the ligature until the ligature-cutting blade reaches the ligature-cutting node and cuts the ligature.

16. The automatic ligature-cutting ligation device as claimed in claim 15, wherein the connecting rod member comprises:
a first connecting rod, an end of the first connecting rod away from the engagement portion being rotatably connected to the sliding member; and
a second connecting rod, an end of the second connecting rod away from the engagement portion being rotatably connected to an end of the first connecting rod close to the engagement portion, wherein the ligature-cutting blade is disposed at an end of the second connecting rod close to the engagement portion.

17. The automatic ligature-cutting ligation device as claimed in claim 15, wherein an end of the elastic member close to the snare is sleeved on the sliding member, and the end of the ligature away from the snare is connected to at least one of the elastic member and the sliding member;
in a case where the elastic member is stretched to a preset pulling force, the ligature-cutting assembly is driven move to the ligature-cutting node and cuts the ligature.

18. The automatic ligature-cutting ligation device as claimed in claim 15, wherein an opening is defined on the second handle, the sliding member is arranged with an extension portion, and the extension portion penetrates out of the opening and protrudes beyond a side wall of the second handle;
or at least a part of the extension portion is disposed in the opening without protruding beyond a side wall of the second handle;
the sliding member comprises a base portion, the base portion is disposed at a side of the extension portion away from the opening and is accommodated in the second handle; and
an end of the elastic member close to the snare is sleeved on the extension portion, and the end of the ligature away from the snare is connected to at least one of the extension portion and the elastic member.

19. The automatic ligature-cutting ligation device as claimed in claim 18, wherein the extension portion comprises a first extension portion and a second extension portion, the first extension portion is disposed at a side of the base portion close to the opening, and the second extension portion extends from the first extension portion in a direction away from the base portion; and
the end of the elastic member close to the snare is sleeved on the first extension portion, and the end of the ligature away from the snare is connected to at least one of the first extension portion and the elastic member.

20. A ligation system, comprising:
an automatic ligature-cutting ligation device, comprising:
a handle assembly, comprising a first handle and a second handle detachably connected to each other;
a catheter assembly, an end of the catheter assembly being connected to the first handle;
a snare, disposed at another end of the catheter assembly, wherein a slit is defined in an inner surface of the snare;
a ligature, penetrating the first handle and the catheter assembly, wherein an end of the ligature is connected to the second handle, another end of the ligature has a loop capable of contracting in a forward direction and stopping in a reverse direction, and the loop is pre-mounted in the snare; and
a ligature-cutting assembly, disposed in the second handle; and
an outer sheath, at least a part of the automatic ligature-cutting ligation device passing through the outer sheath, and the outer sheath being configured to deliver an end of the automatic ligature-cutting ligation device where the snare is disposed into a heart;
wherein in a case where the second handle is separated from the first handle and pulled away from the first handle, the ligature is driven to be moved away from the first handle, and the loop is driven to be pulled out of the snare from the slit to an inner side enclosed by the snare and unidirectionally contract on the target tissue; and
in a case where the second handle is separated from the first handle and pulled away from the first handle until a preset pulling force is reached, the ligature-cutting assembly is driven to move and cut the ligature.
